(19) United States
(12) Patent Application Publication
(10) Pub. No.: US 2020/0020041 A1
(43) Pub. Date: Jan. 16, 2020

(54) APPARATUS, METHOD, AND MEDIUM FOR SURGERY RISK CALCULATION

(71) Applicant: Robert Michael Meneghini, McCordsville, IN (US)

(72) Inventor: Robert Michael Meneghini, McCordsville, IN (US)

(21) Appl. No.: 16/502,563

(22) Filed: Jul. 3, 2019

Related U.S. Application Data
(60) Provisional application No. 62/696,439, filed on Jul. 11, 2018.

(51) Int. Cl. G06Q 40/08; A61B 34/10

(52) U.S. Cl. CPC G06Q 40/08; A61B 34/10

(57) ABSTRACT

An apparatus includes a memory and a processor. The memory stores multiple medical and surgical risk factors. Each medical and surgical risk factor correlates to a possible adverse event or complication occurring during a perioperative period associated with a particular physician or medical institution. The processor performs a risk determination cycle that includes calculating, for the multiple medical and surgical risk factors, individual medical and surgical risk scores, each indicating a likelihood of the possible adverse event or complication for a respective one of the medical and surgical risk factors. The risk determination cycle includes calculating a total medical and surgical risk score by summing the individual medical and surgical risk scores. The risk determination cycle includes calculating a strength of the likelihood of the possible adverse event or complication by comparing the total medical and surgical risk score with scores in a risk range.

### BACKGROUND

The cost of insuring surgeons and medical institutions against malpractice claims is currently inefficient and relatively haphazard due to lack of accurate and granular information by which to assign risk and subsequent cost. This is because at present there is no way of easily determining which surgeons and medical institutions are riskier than others in terms of the likelihood of costly adverse events and complications. While premiums may vary according to non-personal factors such as location and practice area, variance in premiums is not particularly significant unless a malpractice claim against a surgeon or medical institution is actually experienced.

The present description reveals a novel and unique apparatus, method, and medium for determining and evaluating surgical and perioperative risk of surgeons, as well as determining and evaluating risk of medical institutions that provide facilities and support for surgeries. Specifically the likelihood of adverse events and perioperative and surgical complications by a surgeon or facility is determined based on a number of risk factors. Further, the likelihood of adverse events and complications, based on a number of risk factors, on the part of a medical institution that operates a surgical environment and that provides support for a surgeon is also determined.

The likelihood of adverse events and complications of surgeons and/or medical institutions is of course directly correlated to the likelihood of malpractice claims against the surgeons and medical institutions. Identification of higher risks surgeons and higher risk medical institutions will thus result in a more efficient market for medical malpractice insurance as more accurate premiums can be assigned to surgeons and/or institutions based on their specific risk assessment and determination.

### SUMMARY

Accordingly, one embodiment disclosed herein provides an apparatus, configured as a node on a network, comprising a communication interface, a memory, and a processor. The memory stores data representative of a plurality of medical and surgical risk factors. Each medical and surgical risk factor correlates to a possible adverse event or complication occurring during a perioperative period associated with one of a particular physician or a particular medical institution.

The processor performs a risk determination cycle. The risk determination cycle includes calculating for the plurality of medical and surgical risk factors, a plurality of individual medical and surgical risk scores. Each individual medical and surgical risk score indicates a likelihood of the possible adverse event or complication for a respective one of the plurality of medical and surgical risk factors.

The risk determination cycle further includes calculating a total medical and surgical risk score by summing the plurality of individual medical and surgical risk scores. The risk determination cycle lastly includes calculating a strength of the likelihood of the possible adverse event or complication by comparing the total medical and surgical risk score with scores in a risk range previously stored in the memory.

### BRIEF DESCRIPTION OF THE DRAWINGS

The accompanying figures, where like reference numerals refer to identical or functionally similar elements and which together with the detailed description below are incorporated in and form part of the specification, serve to further illustrate various exemplary embodiments and to explain various principles and advantages in accordance with the embodiments.

FIG. 1 is a block diagram illustrating a system for determining and evaluating risk of surgeons and medical institutions.

FIG. 2 is a block diagram illustrating an apparatus for determining and evaluating risk of surgeons and medical institutions.

FIG. 3 is a block diagram illustrating method for determining and evaluating risk of surgeons and medical institutions.

### DETAILED DESCRIPTION

The instant disclosure is provided to further explain in an enabling fashion the best modes of performing one or more embodiments. The disclosure is further offered to enhance an understanding and appreciation for the inventive principles and advantages thereof, rather than to limit in any manner the invention.

It is further understood that the use of relational terms such as first and second, and the like, if any, are used solely to distinguish one from another entity, item, or action without necessarily requiring or implying any actual such relationship or order between such entities, items or actions. It is noted that some embodiments may include a plurality of processes or steps, which can be performed in any order, unless expressly and necessarily limited to a particular order; i.e., processes or steps that are not so limited may be performed in any order.

Much of the inventive functionality and many of the inventive principles when implemented in a processor, are best supported with or in software or integrated circuits (ICs), such as a digital signal processor, and/or application specific ICs. It is expected that one of ordinary skill, notwithstanding possibly significant effort and many design choices motivated by, for example, available time, current technology, and economic considerations, when guided by the concepts and principles disclosed herein will be readily capable of generating such software instructions or ICs with minimal experimentation. Therefore, in the interest of brevity and minimization of any risk of obscuring principles and concepts, further discussion of such software and ICs, if any, will be limited to the essentials with respect to the principles and concepts used by the exemplary embodiments.

The present disclosure presents embodiments for using risk factors to determine and evaluate an individualized risk of adverse events or perioperative complications related to surgery and resulting malpractices claim against particular surgeons and medical institutions, or at a minimum a substantial increase in cost to the insurance provider and the healthcare system as a whole. By analyzing a variety of risk factors, an insurance company can adjust premiums to be more accurately charged to a surgeon or medical institution to reflect the risk of costly adverse events and complications that may result in medical malpractice claims. Additionally, government programs such as Medicare and Medicaid can make determinations as to how much reimbursement should be contributed to a provider and/or whether even certain surgeons and medical institutions should be able to treat patients based on the results of the risk determination.

It should be noted that as used throughout this description and claims, the definition of an “adverse event or complication” is an unexpected or unanticipated medical problem that arises, follows, or is a result of a medical procedure or treatment. Any causing procedure or treatment may or may not be a deviation from accepted medical standards of care. Attention is now given to several of the actual risk factors that are used in determining likelihood of adverse events or perioperative complications related to surgery, and the resulting impacts on insurance premiums.

1. Fellowship Training

As is well known a medical Fellowship is a period of medical training in the United States and Canada that a surgeon may undertake after completing a residency, which is in itself a specialty training program. In various surgical fields, completing a Fellowship provides an advanced level and specialization of training such that completion of a Fellowship tends to minimize the likelihood and probability of adverse events or complications.

It should thus be clear that any surgeon who has completed a Fellowship will be considered to have a lower risk of making errors and mistakes. Further, for medical institutions, the greater the number of surgeons with fellowship training, the lower the risk of adverse events or perioperative complications occurring at the institution. It should also be noted that an additional risk factor or a weighting of the risk factor could also include where a particular Fellowships are conducted.

2. Minimum Annual Surgical Volume

Surgical volume is a measure of a total number of surgeries performed by a surgeon in a given time frame. Similarly, for a medical institution surgical volume is a measure of the number of surgeries performed at the institution. There is sufficient evidence that a minimum number of surgical procedures should be performed by a surgeon and/or institution to maintain proficiency and avoid adverse events or complications related to that surgical procedure. Further, evidence exists that higher volume surgeons and institutions have more optimal outcomes and decreased complication rates.

The typical time frame in which surgical volume is measured is a year (that is, annually). Thus, there is normal range for a competent surgeon or a satisfactory medical institution of the number of surgeries, in any given practice area, that may be performed over a given year. If the number of surgeries performed by a surgeon, or occurring within a medical institution, is outside the lower limit of the range, the risk of errors and mistakes increases due to lack of proficiency and expertise. It should of course be noted that for medical institutions, the range is effected by the number of surgeons on staff and the number of surgical suites available.

3. Maximum Mean Blood Loss

Blood loss during surgery is always a concern. Specifically, some blood loss is always expected during surgery, but excessive blood may be a result of imprecise surgical technique, extra-ordinary soft-tissue trauma, or excessive operative time.

For any particular type of surgery, there is a normal range of blood loss that should occur. If mean blood loss for a particular surgeon is outside the normal range for the particular type of surgery, then there is an increased likelihood of perioperative complications and/or adverse events and corresponding malpractice claim.

It should be noted that blood loss is such a well-established risk factor that an analysis of mean blood loss for a particular surgeon or for a group of surgeons of a particular medical institution must be undertaken with respect to a standard deviation of the normal range. That is to say, if the mean blood loss of a particular surgeon or a group of surgeons of a particular medical institution is outside a single standard deviation of the normal blood loss range, a clear risk is indicated.

4. Maximum Mean Surgical Operative Time

Surgical operative time is also a concern during surgery. Competent surgeons are neither too fast nor too slow in performing surgery. Competent surgeons, for each practice area, and for each type of procedures, complete a surgery within a standard range of time.

If a particular operative time is too fast, this is evidence that a surgeon is hurrying through a procedure for possibly a variety of different reasons. The reasons may include a desire to perform additional surgeries on the same day, a lack of understanding of the complexity of the current surgery being performed, and other professional and/or family pressures.

Similarly, if a particular operative time is too slow, this is evidence of a problem in the surgery for various reasons. The reasons include the surgeon not being skilled enough to perform the operation properly in an acceptable time, the surgeon procrastinating and/or simply not paying attention, and possibly even the surgeon allowing a subordinate employee, such as a resident, to perform the surgery.

It should be noted that a slow operative time relates to another risk factor: anesthesia. It is well understood that the longer a patient is unconscious from general anesthesia, the greater the likelihood of complications either during or after the surgery.

For any particular type of surgery, there is an acceptable range of operative time that should occur. If mean operative time for a particular surgeon is outside the normal range for the particular type of surgery, then there is an increased likelihood of adverse events and complications, and corresponding potential malpractice lawsuits and claims.

It should be noted that mean operative time is such a well-established risk factor that an analysis of mean operative time for a particular surgeon or for a group of surgeons of a particular medical institution must be undertaken with respect to a standard deviation of the normal range. That is to say, if the mean operative time of a particular surgeon or a group of surgeons of a particular medical institution is outside a single standard deviation of the normal operative time range, a clear risk is indicated.

5. 30-day and 90-day Emergency Room Visit Rates

A manner of evaluating success or failure of a surgery is whether a recovering patient experiences further complications upon completion of the surgery. Such complications can result in an emergency room visit by the recovering patient. Thus if something unusual or unexpected occurs during surgery or within the perioperative period, it may result in the patient visiting an emergency room shortly (within 90 days) after the surgery is performed.

Thus 30-day and 90-day emergency room visits are a factor in determining risk of a particular surgeon or group of surgeons in a particular medical institution for further adverse events or complications. As with the other factors, there is a normal range of occurrences of emergency room visits, per a total number of patients, within both 30 and 90 days after surgery. If a number of visits by surgical patients of the particular doctor or group of doctors exceeds the range within the respective time frames, there is an increased probability (greater risk) of further adverse events or complications occurring.

In order to clarify this point, a brief example is provided. Assume that a normal range of patients undertaking emergency room visits within 30 days after hip replacement surgery is between 5-10 patients out of 100 total. The range for emergency room visits within 90 days is assumed to be between 8-13 patients out of 100 total. If a particular doctor or particular group of doctors experiences more than 10 within 30 days and more than 13 within 90 days, this is an indicator that the particular doctor or group of doctors may be experiencing more errors and mistakes than is normal. This increase rate of emergency room visits will signify and indicate increase risk of perioperative adverse outcomes and complications and therefore will factor into the overall risk determination to result in an increase in malpractice or bundle-protection premiums.

6. Readmission Rates

Similar to emergency room visits, readmission rates relate to a condition occurring after surgery is complete. However, readmission after a surgical procedure tend to be more costly due to the inherent adverse event or complication severity that warrants an inpatient admission for successful treatment. Similar to emergency room visits, manner of evaluating success or failure of a surgery is whether a recovering patient experiences further complications upon completion of the surgery that require a readmission to a surgery facility.

Readmission rate is thus an additional factor in determining risk of a particular surgeon or group of surgeons in a particular medical institution. As with the other factors, there is a normal range of occurrences of readmission over a given total number of patients. If the readmission rate for a particular surgeon or group of surgeons exceeds the normal range over a period of 12 months, a there is a higher probability of adverse events or complications occurring.

7. Extended Care/Rehabilitation Facility Placement Rate

Extended care and rehabilitation facilities help transition a patient between hospital care and home. These facilities are used often for elderly patients after surgery. It is known that a greater probability of adverse events or complications will be present if a patient is placed in an extended care rehabilitation facility. Therefore surgeons or institutions which have higher than expected rates of placement in these facilities will subsequently be expected to have greater overall rates of adverse events or complications.

8. Complication Rates

As should be clear, unanticipated complications sometimes occur during surgery. However a higher than average rate of complications would indicate that the complications might be occurring due to perioperative program inadequacies or less than optimal surgeon skill or competence, rather than by chance. Similar to other factors, there is a mean complication rate or range which is considered normal. Complication rates for a particular surgeon, or group of surgeons, that are out of the normal range would indicate an increased probability of adverse events or complications.

It should be noted that the nature of measured complications varies according the many different types of surgery. For example, the complications that occur in orthopedic surgery are much different from the complications arising in neurosurgery or open heart surgery. For example, complications in orthopedic surgery include dislocation of hips and peri-prosthetic fracture around the hip and/or knees. In contrast complications from open heart surgery include heart attack or stroke, irregular heartbeat, and/or lung or kidney failure.

9. Internal Medicine Perioperative Program

Perioperative care is the care that is given immediately before, during, and immediately after surgery, and takes place in hospitals and other surgical centers. There are several sub-factors related to a perioperative program that must be considered to determine the effect of the perioperative program as it relates optimal patient outcomes and minimization of complications after surgery. The risk assessment of perioperative program is a tiered model.

Initially, if there is no medical preoperative assessment, risk of perioperative complications or adverse events increases (Tier 1). Secondly, if the surgeon/program allows each patient's individual family or internal medicine physician to perform preoperative assessment, clearance, and optimization (Tier 2), there will be a larger degree of variability in the quality and consistency of perioperative medical care, subsequently increasing the probability of adverse events of medically related complications, relative to the Tier 1 methodology. In a third tier, if pre-admission testing is performed by a large medical group that rotates a large number of internal medicine and anesthesiologists (Tier 3), a relative lack of familiarity with the patient may lead to less informed decisions and inconsistency of personnel may result in increased risks of complications relative to a Tier 4 below, but would be less risk compared to Tier 2. Lastly, if a perioperative program is simply a small group of consistent perioperative medical specialists who use relatively standardized and evidence-based protocols (Tier 4), this tends to decrease risk and minimize the probability of adverse events or complications.

It should be noted that there are applications and programs for assessing whether particular patients are good candidates for certain types of surgery. If any perioperative program includes such an application for easily determining whether a patient is a good candidate for a proposed surgery, such a perioperative program may indicate a lower probability and risk of adverse events or complications occurring in the perioperative period.

10. Implementations of New Technology

Use of new technology in surgery is a factor in determining risk of a particular surgeon or group of surgeons in a particular medical institution. Specifically, adoption of new technology such as a new surgical approach, new surgical instrumentation, etc. may increase the risk of adverse events and complications during the “learning curve” of that technology adoption. There are certain technologies or techniques that have learning curves of varying character and duration that are associated with a greater risk of complications during the time a surgeon or institution is becoming familiar with the techniques. Therefore, during the period of adoption, the surgeon and/or facility will be considered to have a greater probability of adverse events or complications.

11. Multi-Modal Pain Program

Adequate pain management is very important to the recovery of patients after various types of surgery, including many orthopedic surgeries. An effective multi-modal pain management system decreases narcotic requirements, and results in less pain with fewer side effects. Conversely, inadequate pain control has been shown to adversely affect patient outcome and increase the probability of adverse outcomes and complications. Patients of particular surgeons and particular groups of surgeons of medical institutions that have an effective multi-modal pain program are less likely to experience adverse outcomes and complications, and as such, are less likely to experience a malpractice claim.

12. Anesthesia Program

It should be easily understood that the competent application of anesthesia during surgery is of utmost importance. Complications and significant problems from faulty administration of anesthesia are well known. Thus, the quality of the anesthesia program at medical institutions is an important factor in determining risk of a medical institution in errors and mistakes, as well as adverse outcomes and complications during the perioperative period.

In determining the quality of an anesthesia program, the variance and consistency of practitioners in a medical institution are reviewed. If the same anesthesiologists and nurse anesthetists consistently work with the same surgeons, familiarity leads to better cooperation such that the likelihood of errors and mistakes is decreased and patient outcomes are optimized.

It should also be noted that the volume of surgeries performed by anesthesiologists and nurse anesthetists in a medical institution is also reviewed in determining quality of an anesthesia program. Similar to the general risk from excessive volume discussed above, anesthesiologists and nurse anesthetists that perform a sufficient number of hip and knee related procedures will typically have a lower risk of perioperative complications, better pain control, and less risk of adverse events.

13. Pre-Operative Teaching and Patient Education Program

It should be clear that the more informed a patient and their family is about an impending surgical procedure, the more likely that the patient can aid in making the surgery proceed more comfortably and without errors or complications. That is to say, an informed patient can more easily discuss potential problems or issues as they arise rather than ignoring any potential problems.

Because of the above benefit, a high-quality pre-operative patient teaching and education program is considered to reduce the risk of a surgeon, or group of surgeons in a medical institution, experiencing adverse events and perioperative complications. In addition, outlining appropriate expectations for the outcome of surgery, as well as a thorough discussion of the risk of complications and adverse events, will lead to greater probability of having a patient that is satisfied when their expectations are met, and who is understanding if a complication or adverse event occurs, thereby reducing the probability the patient will file a malpractice suit. A medical institution is also evaluated for risk by whether it operates a pre-operative patient teaching and education program, and if so, of what quality is the program.

14. Historical Malpractice Claims Rates

As noted above, the risk analysis disclosed herein is not undertaken merely to determine high-quality surgeons from other surgeons. Rather the analysis of the risk of errors and mistakes is to determine how to price malpractice premiums for individual surgeons and medical institutions. However, determining malpractice insurance premiums is not only a function of past surgical variables, but of actual past malpractice insurance claims.

Of course actual past malpractice claims are a result of previous errors and mistakes, so to some extent they are indicators of possible future errors and mistakes. Thus a review of historical malpractice claims and claims rates is undertaken for a particular surgeon or group of surgeons in a medical institution to determine the likelihood of future errors and mistakes and the corresponding increase in malpractice premiums.

15. Malpractice Risk Mitigation Training Modules

Surgeons should regularly and satisfactorily complete courses and education materials aimed at teaching healthcare providers how to minimize risk and improve patient safety, as well as minimize risk that could result in a malpractice lawsuit or claim. As should be expected, participation in these malpractice risk mitigation training modules would reduce the risk of errors and mistakes on the part of any surgeon or group of surgeons at a medical institution.

16. Board Certification

When surgeons graduate from medical school, and do an appropriate residency, they are eligible for licensure. This is a state-issued permit to practice medicine that means minimal training and competency requirements have been met. Board certification further implies that the practitioner has gone above and beyond that minimal standard in a particular specialty or subspecialty by way of extra education and study. The surgeon must further pass an examination to prove his or her competence in specialty in which the surgeon is to be board certified. As well, some licensing boards require that a surgeon have been practicing medicine for a certain number of years before becoming eligible for board certification.

In terms of risk assessment, obtaining board certification by a surgeon is an indicator that the surgeon is less likely to experience adverse events and complications compared to those who have not obtained board certification. Thus, obtaining board certification indicates a surgeon is less likely to be the subject of a medical malpractice claim.

17. Annual Certified Medical Education (CME) Credits

All surgeons are required to complete a certain number of hours of ongoing scholarly pursuit, known as certified medical education, or CME, in order to maintain licensure. CME participation allows a surgeon to keep current in the latest techniques in his/her specialty. CME credits are mandated by the licensing board of each state in which the surgeon is licensed. Being deficient in annual CME credits would certainly represent an increased probability of adverse events or perioperative complications compared to those who attain a sufficient quantity of continuing medical education.

18. Membership in Professional Organizations

Although surgeons are required to be licensed by the governing boards of states in which they practice, membership in professional organizations is voluntary. Professional organizations provide intangible benefits and resources that aid a surgeon in optimizing patient care, including peer conferences, medical journals, telephone hotlines, and the like. As these benefits can contribute to the surgeon's overall knowledge base, as well as enhance an ability to enact optimal surgical and perioperative outcomes, voluntary membership in professional organizations is generally an indicator that the surgeon will be less likely to experience perioperative adverse events or complications.

Each surgical specialty has its own professional organizations. For example, and related to orthopedic surgery, some well-known professional organizations are: American Association of Hip and Knee Surgery (AAHKS); International Congress for Joint Reconstruction (ICJR); American Association of Orthopedic Surgeons (AAOS); and the prestigious Hip Society and Knee Society, both requiring election into the organization based on evidence and reputation as a national thought leader in the field.

It should be noted that if a particular surgeon is not only a voluntary member of a professional organization, but actually further takes a leadership position in the organization, the particular surgeon is even less likely to experience perioperative adverse events or complications. Therefore, being an executive board member or an officer of a professional organization should result in lower medical malpractice insurance premiums.

19. Teaching

Another risk factor effecting whether a medical institution experiences adverse outcomes and complications relates to whether the institution is a teaching institution. A resident, as mentioned above, is a surgeon who practices medicine under the direct or indirect supervision of an attending physician. If a teaching institution maintains a high number of residents working therein, or relies too heavily on the residents in providing medical care, including surgery, there is a higher probability of adverse outcomes and complications. Further, the probability of adverse events or complications is greater if a surgeon gives a resident more autonomy, compared to those surgeons who provide a greater level of supervision with a more “hands-on” approach.

As discussed above, teaching institutions also provide Fellowships to doctors seeking advanced training. Although a high percentage of Fellows are fairly new physicians, it has been demonstrated that the presence of a Fellowship program at a teaching institution may lower the probability of adverse outcomes and complications. This result may be related to the fact that Fellows tend to be at a more advanced training level focused within a surgical subspecialty.

20. Infection Prevention Program

An infection prevention program is an invaluable tool in warding off complications from infection after surgery. Infection is particularly problematic in orthopedic surgery where foreign implants are the norm. Those medical institutions that offer an infection prevention program, developed in accord with established protocol, have a lower probability of experiencing adverse outcomes and complications.

21. Patient Satisfaction Rates

Although sometimes adverse outcomes and complications do occur, if a patient is happy about his/her selection of a surgeon, malpractice claims can be avoided. Thus it is important for surgeons, and all physicians for that matter, to keep in mind the emotional and social needs of a patient. Communication and empathy, combined with surgeon skill, can go a long way in keeping patients satisfied. Thus, patient satisfaction rates are an indicator that a surgeon or medical institution will not experience adverse outcomes and complications and malpractice claims resulting therefrom.

22. Personal Communication Protocol

There is one action taken by surgeons that is well known both to increase patient satisfaction and decrease adverse outcomes and complications: the surgeon answering his own patient after-hours calls. That is to say, when the surgeon personally takes calls from patients, the patient is both more informed and more satisfied by being more connected directly with their performing surgeon. Being informed and connected to their surgeon of course allows the patient to better understand any potential adverse outcomes and complications after a surgical procedure, and being satisfied may aid in the patient foregoing retaliatory action in the event of any kind of adverse outcome or complication.

It should be noted that there are software programs that aid a surgeon in improving communication with patients. “Connected medicine” programs such as Force Therapeutics, Twissle, and others help keep the surgeons actively communicating with patients and help patients stay engaged in their health care. In summary, using communication protocols that in some ways force a surgeon to fully communicate with patients deceases the probability of both adverse outcomes and complications, and further decreases the probability of malpractice litigation.

23. Geographic Region of Practice

As indicated above, in certain geographic locations there are an increased number of adverse outcomes and complications that result in increased malpractice claims. Thus even though it is not necessarily related to actual performance, a surgeon's or institution's geographic location is an indicator of adverse outcomes and complications.

24. Emergency Room Call Coverage

Generally speaking, any surgeon that works on a regular basis in an emergency room has an increased likelihood of adverse outcomes and complications. Specifically, the wide array of different procedures that must be performed and the extremely expedited nature of surgical procedures in the emergency room are factors that give rise to adverse outcomes and complications. There are different types of emergency room call coverage scenarios, each type indicating a different risk of adverse outcomes and complications: no ER call whatsoever; Level 1 with trauma colleagues available; Level 1 without trauma team/colleagues available; and Level 2 or greater.

25. Specialty Specific Factors

Each particular surgery specialty has its own factors that increase the likelihood of adverse outcomes and complications, and as well, for each surgical specialty there are actions that can be taken to decrease the risk of adverse outcomes and complications. For example, in orthopedic surgery in a total hip arthroplasty, lengthening of one leg over the other can occur if the surgeon has not properly planned the surgery. In total hip arthroplasty, surgeon templating and/or using intra-operative radiography mitigates the likelihood of leg lengthening. Such action by the orthopedic surgeon decreases the risk of adverse outcomes and complications.

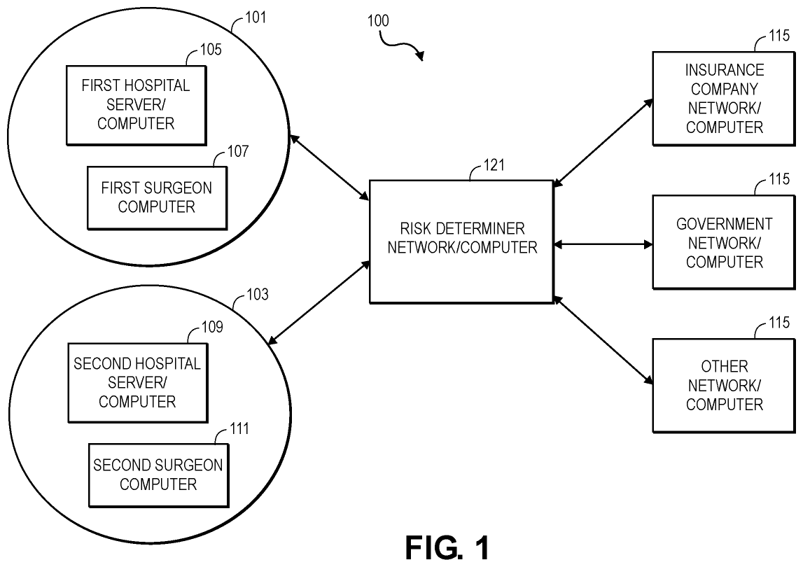

Now that an overview of several risk factors has been presented, a system that determines and evaluates risk of surgeons and medical institutions that provide facilities and support for surgeries is discussed and described. FIG. 1 is a block diagram illustrating the system 100 for determining and evaluating risk of surgeons and medical institutions (further denoted as a surgery risk determination system 100). The surgery risk determination system 100 includes a first hospital network 101, a second hospital network 103, an intermediary risk determiner network 121, an insurance company network 115, a government network 117, and an other network 119 (including for example, a network of a prospective patient, prospective employer, or journalist). The first hospital network 101 includes a first hospital server 105 and a first surgeon computer 107. The second hospital network 103 includes a second hospital server 109 and a second surgeon computer 111.

Since surgeons often use hospital facilities to perform surgeries, the hospital servers 105, 109 and the surgeons 107, 111 are viewed as being part of respective hospital networks 101, 103. However, it should be understood that when evaluating risk, a surgeon 107, 111 may be judged independently or he/she may be judged as part of the overall hospital as an institution.

The hospital servers 105, 109 and the surgeon computers 107, 111 may respectively communicate over a local area network (LAN), or if enough surgeons and other devices are interconnected, a wide area network (WAN). Succinctly put, surgeons and hospitals typically exchange significant amount of data with each other.

The risk determiner network 121 is the actual mechanism for determining risk of adverse events and complications of a surgeon or group of surgeons in a medical institution. The risk determiner network 121 is programmed and/or configured to use the twenty-five risk factors discussed above to determine the probability of adverse events and complications.

Such a determination by the risk determiner network 121 is in the form of an adverse event, complication, and malpractice risk score. The adverse event, complication, and malpractice risk scores may be calculated by the risk determiner network 121 by determining a risk numerical score for each of the twenty-five risk factors described above, and simply summing the risk numerical scores. In an alternative embodiment, one or more the twenty-five risk factors above may be weighted by the risk determiner network 121 to reflect greater or less importance. The weighted risk numerical scores are then totaled by the risk determiner network 121 to determine the adverse event, complication, and malpractice risk score.

Once the adverse event, complication, and malpractice risk score is obtained, the risk determiner network 121 compares the adverse event, complication, and malpractice risk with a previously established risk range. A score in one part of the risk range will indicate a very high likelihood of adverse events and complications and a very high likelihood of a malpractice claim. A score in a second part of the risk range will indicate a moderately high likelihood of adverse events and complications and a moderately high likelihood of a malpractice claim. A score in a third part of the risk range will indicate a moderately low likelihood of adverse events and complications and a moderately low likelihood of a malpractice claim. A score in a fourth part of the risk range will indicate a very low likelihood of adverse events and complications and a very low likelihood of a malpractice claim. It should be noted that the four-part risk range described above is exemplary, and the risk range could be broken down into many different parts with corresponding likelihoods.

Once the risk determiner network 121 determines the adverse event, complication, and malpractice risk, and determines where in the risk range the score falls, the risk determiner network 121 can transmit this information to any of the insurance company network 115, the government network 117, or the other network 119. As discussed above, the insurance company network 115 can take the result of the determinations by the risk determiner network 121 and calculate a malpractice insurance premium based on the results.

It needs to be pointed out that the embodiment of FIG. 1, where the risk determiner network 121 is an intermediary network, is merely exemplary. The determination of the adverse event, complication, and malpractice risk score could be performed within each of the insurance company network 115, the government network 117, or the other network 119 using an application or a separate computing mechanism. That is to say, the risk determining capability can be configured in several different ways known in the art, and is not limited to that specifically disclosed herein.

It should be clear that there is a direct correspondence between premiums and risk score as the risk scores moves in the risk range from very low likelihood of adverse events and complications to very high likelihood of adverse events and complications. That is malpractice premiums are at their lowest when there is a very low likelihood of adverse events and complications and at their highest when there is a very high likelihood of adverse events and complications.

It should be noted that the risk assessment methodology outlined herein may also be used to determine the probability of a surgeon and/or surgery program of having an adverse event or medical complication in the perioperative period that could result in excessive costs. An adverse event or medical complication that results in excessive cost can be referred to as a “Bundle Buster.”

Bundling is a compensation technique used by private insurers and the government whereby one payment is made for aggregated services provided over a certain period of time. Bundling can be thought of as “flat fee” payment for the aggregated services. A “Bundle Buster” occurs when an adverse event or complication causes cost to rise significantly over the typical flat fee amount, and the insurer or government entity is forced to pay additional costs. It should be clear that an insurer or government entity will try to determine surgeons and medicals institutions that have a lower probability of experiencing a Bundle Buster.

MEDICAID and MEDICARE as well as other state and local entities are represented in FIG. 1 as the government network 117. The government network 117 can also use the results obtained by the risk determiner network 121 including the determined malpractice risk score and its location in the risk range. The government network 117 will calculate payment rates to surgeons, selection of surgeons, and selection of hospitals, as just a few examples, based on the results of the risk determiner network 121.

In FIG. 1, the other network 119 represents any number of other entities that might have an interest in obtaining and processing the results of the risk determiner network 121. Such entities might be non-profit consumer affairs agencies, journalists, and medical employers. It should be noted that whether it is the other network 119, or the government network 117, or the insurance company network 115 that obtains the results of the risk determiner network 121, further processing by the obtaining network will produce an additional result of some sort. For example, the insurance company network 115 will use the determined malpractice risk score and its location in the risk range, obtained from the risk determiner network 121, to calculate an appropriate malpractice insurance premium.

It should be noted that the communication arrangement between the first and second hospital networks 101, 103, the risk determiner network 121, the insurance company network 115, the government network 117, and the other network 19 is a significant improvement in technology over prior art systems. Specifically, the single processing path, indicated by the transfer of data from the hospital networks 101, 103 to the risk determiner network 121, the processing of the malpractice risk score and determination of where in the risk range the risk score lays by the risker determiner network 121, and the further transfer of these results to one or more of the insurance company network 115, the government network 117, and the other network 19 for further processing, creates a tremendous resource efficiency not achieved by the prior art. Where networked apparatuses communicate and process data in a single processing path rather than plural processing paths, as in the prior art, tremendous efficiency of resources is achieved.

Each of the first and second hospital networks 101, 103 including the first and second hospital servers 105, 109 and the first and second surgeon computers 107, 111, the risk determiner network 121, the insurance company network 115, the government network 117, and the other network 19 may be viewed as a computer system or network. The computer systems/networks 101, 103, 105, 107, 109, 111, 121, 115, 117, 119 may communicate each with the other over any network such as the Internet, an intranet, or any other network. Each computer system/network 101, 103, 105, 107, 109, 111, 121, 115, 117, 119 may be programmed to operate in automated fashion, and may also have an analog or a graphic user interface such as Outlook and Windows such that users can control computer systems 103, 105, 107, 111, 113, 115, 117. Each computer system/network 101, 103, 105, 107, 109, 111, 121, 115, 117, 119 may include at least a central processing unit (CPU) with data storage such as disk drives, the number and type of which are variable. In each computer system/network 101, 103, 105, 107, 109, 111, 121, 115, 117, 119 there might be one or more of the following: a floppy disk drive, a hard disk drive, a solid state drive, a CD ROM or digital video disk, or other form of digital recording device.

Each computer system/network 101, 103, 105, 107, 109, 111, 121, 115, 117, 119 may include one or more displays upon which information may be displayed. Input peripherals, such as a keyboard and/or a pointing device, such as a mouse, may be provided in each computer system/network 101, 103, 105, 107, 109, 111, 121, 115, 117, 119 as input devices to interface with each respective CPU. To increase input efficiency, the keyboard may be supplemented or replaced with a scanner, card reader, or other data input device. The pointing device may be a mouse, touch pad control device, track ball device, or any other type of pointing device.

Each computer system/network 101, 103, 105, 107, 109, 111, 121, 115, 117, 119 may interconnect peripherals previously mentioned herein through a bus supported by a bus structure and protocol. The bus may serve as the main source of communication between components of each computer system/network 101, 103, 105, 107, 109, 111, 121, 115, 117, 119. The bus in each computer system/network 101, 103, 105, 107, 109, 111, 121, 115, 117, 119 may be connected via an interface.

The CPU of each computer system/network 101, 103, 105, 107, 109, 111, 121, 115, 117, 119 may perform the calculations and logic operations required to execute the functionality of each computer system as described in this disclosure. The functionality of each computer system/network 101, 103, 105, 107, 109, 111, 121, 115, 117, 119 may be processed in an automated fashion such that relevant data is processed without user administrator assistance or intervention. Alternatively or additionally, the functionality of each computer system/network 101, 103, 105, 107, 109, 111, 121, 115, 117, 119 may be processed in a semi-automatic fashion with intervention from a user administrator at one or more of the computer systems/networks 101, 103, 105, 107, 109, 111, 121, 115, 117, 119. Implementing, processing, and executing the functionality of each computer system/network 101, 103, 105, 107, 109, 111, 121, 115, 117, 119 as described in this disclosure is within the purview and scope of one of ordinary skill in the art, and is not discussed in detail herein.

Each computer system/network 101, 103, 105, 107, 109, 111, 121, 115, 117, 119 may be implemented as a distributed computer system or a single computer. Similarly, each computer system/network 101, 103, 105, 107, 109, 111, 121, 115, 117, 119 may be a general purpose computer, or a specially programmed special purpose computer. Moreover, processing in each computer system/network 101, 103, 105, 107, 109, 111, 121, 115, 117, 119 may be controlled by a software program on one or more computer systems or processors, or could even be partially or wholly implemented in hardware. The computer systems/networks 101, 103, 105, 107, 109, 111, 121, 115, 117, 119 used in connection with the functionality may rely on the integration of various components including, as appropriate and/or if desired, hardware and software servers, database engines, and/or other content providers.

Although the computer systems/networks 105, 107, 109, 111, 121, 115, 117, 119 in FIG. 1 are illustrated as being a single computer, each computer system according to one or more embodiments of the invention is optionally suitably equipped with a multitude or combination of processors or storage devices. For example, each computer illustrated in computer systems 105, 107, 109, 111, 121, 115, 117, 119 may be replaced by, or combined with, any suitable processing system operative in accordance with the principles of embodiments of the present disclosure, including sophisticated calculators, hand-held smart phones, smartpads, laptop/notebook, mini, mainframe and super computers, as well as processing system network combinations of the same. Further, portions of each computer system/network 101, 103, 105, 107, 109, 111, 121, 115, 117, 119 may be provided in any appropriate electronic format, including, for example, provided over a communication line as electronic signals, provided on floppy disk, provided on CD-ROM, provided on optical disk memory, etc.

Any presently available or future developed computer software language and/or hardware components can be employed in the computer systems/networks 101, 103, 105, 107, 109, 111, 121, 115, 117, 119. For example, at least some of the functionality mentioned above could be implemented using Visual Basic, C, C++ or any assembly language appropriate in view of the processor being used. It could also be written in an interpretive environment such as Java and transported to multiple destinations to various users.

It is likely that one or more of the computer systems/networks 101, 103, 105, 107, 109, 111, 121, 115, 117, 119 may be implemented on a web based computer, e.g., via an interface to collect and/or analyze data from many sources. User interfaces may be developed in connection with an HTML display format, XML, or any other mark-up language known in the art. It is possible to utilize alternative technology for displaying information, obtaining user instructions and for providing user interfaces.

As indicated above, each computer system/network 101, 103, 105, 107, 109, 111, 121, 115, 117, 119 may be connected over the Internet, an intranet, or over a further network. Links to any network may be a dedicated link, a modem over a POTS line, and/or any other method of communicating between computers and/or users.

Each computer system/network 101, 103, 105, 107, 109, 111, 121, 115, 117, 119 may store collected information in a database. An appropriate database may be on a standard server, for example, a small Sun™ Sparc™ or other remote location. The information may, for example, optionally be stored on a platform that may, for example, be UNIX-based. The various databases may be in, for example, a UNIX format, but other standard data formats may be used. The database optionally is distributed and/or networked.

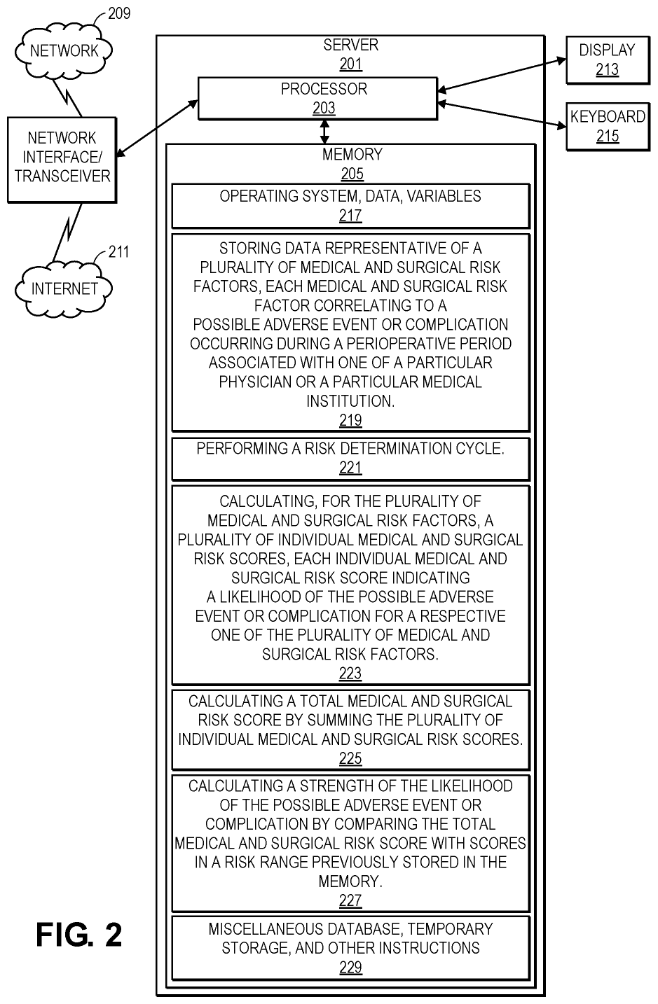

FIG. 2 is a block diagram illustrating an apparatus 201 for determining and evaluating risk of surgeons and medical institutions. In the illustrated embodiment, the apparatus 201 is in particular a surgery risk determination server 201. The surgery risk determination server 201 configured to implement a surgery risk determination is now discussed and described.

The surgery risk determination server 201 may include a transceiver 207, a processor 203, a memory 205, a display mechanism 213, and a keypad and/or touch screen 215. The transceiver 207 may be equipped with a network interface that allows the surgery risk determination server 201 to communicate with other devices in a physician/hospital/insurance/government or other network 209 or over the Internet 211. Alternatively, the network interface may be provided in a separate component coupled with the transceiver 207.

The processor 203 may comprise one or more microprocessors and/or one or more digital signal processors. The memory 205 may be coupled to the processor 203 and may comprise a read-only memory (ROM), a random-access memory (RAM), a programmable ROM (PROM), and/or an electrically erasable read-only memory (EEPROM). The memory 205 may include multiple memory locations for storing, among other things, an operating system, data and variables 217 for computer programs executed by the processor 203.

The computer programs cause the processor 203 to operate in connection with various functions as now described. A storing medical and surgical risk factors function 219 causes the processor 203 to further cause the memory 205 to store data representative of a plurality of medical and surgical risk factors, each medical and surgical risk factor correlating to a possible adverse event or complication occurring during a perioperative period associated with one of a particular physician or a particular medical institution. A risk determination cycle function 221 causes the processor 203 to perform a risk determination cycle.

The risk determination cycle function 221 further includes a calculating a plurality of individual medical and surgical risk scores function 223, a calculating a total medical and surgical risk score function 225, and a calculating a strength function 227. The calculating a plurality of individual medical and surgical risk scores function 223 causes the processor 203 to calculate for the plurality of medical and surgical risk factors, a plurality of individual medical and surgical risk scores, each individual medical and surgical risk score indicating a likelihood of the possible adverse event or complication for a respective one of the plurality of medical and surgical risk factors.

The calculating a total medical and surgical risk score function 225 causes the processor 203 to calculating a total medical and surgical risk score by summing the plurality of individual medical and surgical risk scores. The calculating a strength of likelihood function 227 causes the processor 203 to calculate the strength of the likelihood of the possible adverse event or complication by comparing the total medical and surgical risk score with scores in a risk range previously stored in the memory.

The above described functions stored as computer programs may be stored, for example, in ROM or PROM and may direct the processor 203 in controlling the operation of the surgery risk determination server 201. The memory 205 can additionally store a miscellaneous database and temporary storage 229 for storing other data and instructions not specifically mentioned herein.

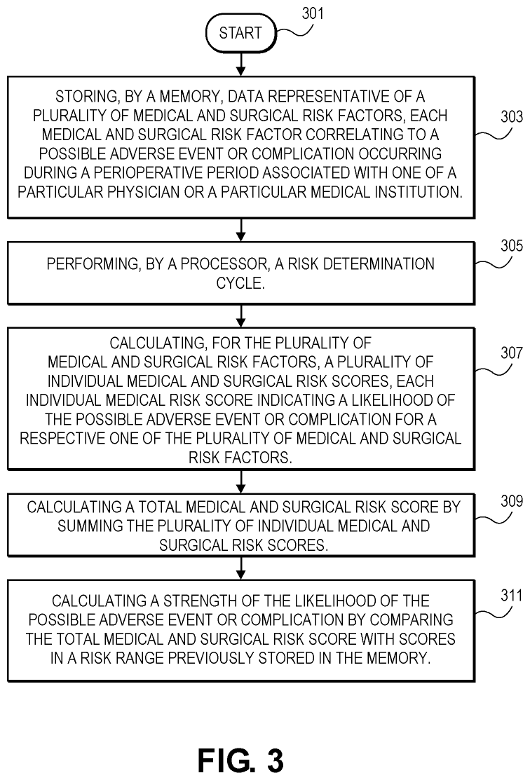

Turning now to FIG. 3, a flow chart illustrating a method for determining and evaluating risk of surgeons and medical institutions, further referred to herein as a surgery risk determination method, is discussed and described. The surgery risk determination method is advantageously implemented in a surgery risk determination apparatus configured as a node on a network, the surgery risk determination apparatus including a communication interface, a memory, and a processor. When one or more of an insurer, a government entity, a prospective patient, a prospective employer, and a journalist decides to acquire information regarding a surgical and perioperative risk of one or more surgeons and/or medical institutions that provide facilities and support for surgeries, the surgery risk determination method begins at 301.

The method comprises storing 303, by the memory, data representative of a plurality of medical and surgical risk factors, each medical and surgical risk factor correlating to a possible adverse event or complication occurring during a perioperative period associated with one of a particular physician or a particular medical institution. The method further comprises performing 305, by the processor, a risk determination cycle.

The risk determination cycle performed 305 by the processor further includes calculating 307, for the plurality of medical and surgical risk factors, a plurality of individual medical and surgical risk scores, each individual medical and surgical risk score indicating a likelihood of the possible adverse event or complication for a respective one of the plurality of medical and surgical risk factors. The risk determination cycle performed 305 by the processor also includes calculating 309 a total medical and surgical risk score by summing the plurality of individual medical and surgical risk scores. The risk determination cycle performed 305 by the processor lastly includes calculating 311 a strength of the likelihood of the possible adverse event or complication by comparing the total medical and surgical risk score with scores in a risk range previously stored in the memory.

This disclosure is intended to explain how to fashion and use various embodiments in accordance with inventive principles, rather than to limit the true, intended, and fair scope and spirit thereof. The foregoing description is not intended to be exhaustive or to limit the inventive principles to the precise form disclosed. Modifications or variations are possible in light of the above teachings. The embodiments disclosed and discussed herein were chosen and described to provide the best illustration of the inventive principles and the practical application thereof, and to enable one of ordinary skill in the art to utilize the inventive principles in various embodiments and with various modifications as are suited to the particular use contemplated. All such modifications and variations should be deemed to be within the scope of the inventive principles disclosed herein.

What is claimed is:

1. An apparatus, configured as a node on a network, comprising:
a communication interface;
a memory storing data representative of a plurality of medical and surgical risk factors, each medical and surgical risk factor correlating to a possible adverse event or complication occurring during a perioperative period associated with one of a particular physician or a particular medical institution; and
a processor performing a risk determination cycle, including:
calculating for the plurality of medical and surgical risk factors, a plurality of individual medical and surgical risk scores, each individual medical and surgical risk score indicating a likelihood of the possible adverse event or complication for a respective one of the plurality of medical and surgical risk factors,
calculating a total medical and surgical risk score by summing the plurality of individual medical and surgical risk scores, and
calculating a strength of the likelihood of the possible adverse event or complication by comparing the total medical and surgical risk score with scores in a risk range previously stored in the memory.

2. The apparatus of claim 1, wherein:
the processor calculating the plurality of individual medical and surgical risk score includes, for each individual medical and surgical risk score, further calculating a product of the individual medical and surgical risk score and a respective weight, the respective weight correlating to an importance of the respective one of the plurality of medical and surgical risk factors relative to a remainder of the plurality of medical and surgical risk factors.

3. The apparatus of claim 1, wherein
the plurality of medical and surgical risk factors includes one or more of the following:
Fellowship Training, Minimum Annual Surgical Volume, Maximum Mean Blood Loss, Maximum Mean Surgical Operative Time, 30-day and 90-day Emergency Room Visit Rates, Readmission Rates, Extended Care/Rehabilitation Facility Placement Rate, Complication Rates, Internal Medicine Perioperative Program, Implementations of New Technology, Multi-Modal Pain Program, Anesthesia Program, Pre-Operative Teaching and Patient Education Program, Historical Malpractice Claims Rates, Malpractice Risk Mitigation Training Modules, Board Certification, Annual Certified Medical Education (CME) Credits, Membership in Professional Organizations, Medical Teaching, Infection Prevention Program, Patient Satisfaction Rates, Personal Communication Protocol, Geographic Region of Practice, Emergency Room Call Coverage, and Specialty Specific Factors.

4. The apparatus of claim 1, wherein
the processor performs the risk determination cycle in response to an electronic request that is received at the interface from an apparatus, configured as a node on a remote network, that is operated by one or more of an insurer, a government entity, a prospective patient, a prospective employer, and a journalist, and
the processor transmits the strength of the likelihood of the possible adverse event or complication through the communication interface to the apparatus operated by the one or more of the insurer, the government entity, the prospective patient, the prospective employer, and the journalist.

5. The apparatus of claim 4, wherein
the electronic request is received from the insurer, and
after transmitting the strength of the likelihood of the possible adverse event or complication to the apparatus operated by the insurer, the processor further:
calculates a malpractice premium amount for the particular physician or the particular medical institution based on the strength of the likelihood of the possible adverse event or complication, and
transmits, through the communication interface, the malpractice premium amount to the apparatus operated by the insurer.

6. A method, implemented in an apparatus configured as a node on a network, the apparatus including a communication interface, a memory, and a processor, the method comprising:
storing, by the memory, data representative of a plurality of medical and surgical risk factors, each medical and surgical risk factor correlating to a possible adverse event or complication occurring during a perioperative period associated with one of a particular physician or a particular medical institution; and
performing, by the processor, a risk determination cycle, including:
calculating, for the plurality of medical and surgical risk factors, a plurality of individual medical and surgical risk scores, each individual medical and surgical risk score indicating a likelihood of the possible adverse event or complication for a respective one of the plurality of medical and surgical risk factors,
calculating a total medical and surgical risk score by summing the plurality of individual medical and surgical risk scores, and
calculating a strength of the likelihood of the possible adverse event or complication by comparing the total medical and surgical risk score with scores in a risk range previously stored in the memory.

7. The method of claim 6, wherein:
calculating the plurality of individual medical and surgical risk score includes, for each individual medical and surgical risk score, further calculating a product of the individual medical and surgical risk score and a respective weight, the respective weight correlating to an importance of the respective one of the plurality of medical and surgical risk factors relative to a remainder of the plurality of medical and surgical risk factors.

8. The method of claim 6, wherein
the plurality of medical and surgical risk factors includes one or more of the following:
Fellowship Training, Minimum Annual Surgical Volume, Maximum Mean Blood Loss, Maximum Mean Surgical Operative Time, 30-day and 90-day Emergency Room Visit Rates, Readmission Rates, Extended Care/Rehabilitation Facility Placement Rate, Complication Rates, Internal Medicine Perioperative Program, Implementations of New Technology, Multi-Modal Pain Program, Anesthesia Program, Pre-Operative Teaching and Patient Education Program, Historical Malpractice Claims Rates, Malpractice Risk Mitigation Training Modules, Board Certification, Annual Certified Medical Education (CME) Credits, Membership in Professional Organizations, Medical Teaching, Infection Prevention Program, Patient Satisfaction Rates, Personal Communication Protocol, Geographic Region of Practice, Emergency Room Call Coverage, and Specialty Specific Factors.

9. The method according to claim 6, wherein:
performing the risk determination cycle occurs in response to an electronic request that is received at the interface from an apparatus, configured as a node on a remote network, that is operated by one or more of an insurer, a government entity, a prospective patient, a prospective employer, and a journalist, and
the method further comprises transmitting, by the processor, the strength of the likelihood of the possible adverse event or complication through the communication interface to the apparatus operated by the one or more of the insurer, the government entity, the prospective patient, the prospective employer, and the journalist.

10. The method of claim 9, wherein
the electronic request is received from the insurer, and
the method further comprises, after transmitting the strength of the likelihood of the possible adverse event or complication to the apparatus operated by the insurer:
calculating, by the processor, a malpractice premium for the particular physician or the particular medical institution based on the strength of the likelihood of the possible adverse event or complication, and
transmitting, by the processor through the communication interface, the malpractice premium to the apparatus operated by the insurer.

11. A non-transitory computer-readable storage medium with instructions stored thereon, that when executed by an apparatus, including a communication interface, a memory, and a processor, cause the apparatus to perform a method comprising:
storing, by the memory, data representative of a plurality of medical and surgical risk factors, each medical and surgical risk factor correlating to a possible adverse event or complication occurring during a perioperative period associated with one of a particular physician or a particular medical institution; and
performing, by the processor, a risk determination cycle, including:
calculating, for the plurality of medical and surgical risk factors, a plurality of individual medical and surgical risk scores, each individual medical and surgical risk score indicating a likelihood of the possible adverse event or complication for a respective one of the plurality of medical and surgical risk factors,
calculating a total medical and surgical risk score by summing the plurality of individual medical and surgical risk scores, and
calculating a strength of the likelihood of the possible adverse event or complication by comparing the total medical and surgical risk score with scores in a risk range previously stored in the memory.

12. The non-transitory computer-readable storage medium according to claim 11, wherein
calculating the plurality of individual medical and surgical risk score includes, for each individual medical and surgical risk score, further calculating a product of the individual medical and surgical risk score and a respective weight, the respective weight correlating to an importance of the respective one of the plurality of medical and surgical risk factors relative to a remainder of the plurality of medical and surgical risk factors.

13. The non-transitory computer-readable storage medium according to claim 11, wherein
the plurality of medical and surgical risk factors includes one or more of the following:
Fellowship Training, Minimum Annual Surgical Volume, Maximum Mean Blood Loss, Maximum Mean Surgical Operative Time, 30-day and 90-day Emergency Room Visit Rates, Readmission Rates, Extended Care/Rehabilitation Facility Placement Rate, Complication Rates, Internal Medicine Perioperative Program, Implementations of New Technology, Multi-Modal Pain Program, Anesthesia Program, Pre-Operative Teaching and Patient Education Program, Historical Malpractice Claims Rates, Malpractice Risk Mitigation Training Modules, Board Certification, Annual Certified Medical Education (CME) Credits, Membership in Professional Organizations, Medical Teaching, Infection Prevention Program, Patient Satisfaction Rates, Personal Communication Protocol, Geographic Region of Practice, Emergency Room Call Coverage, and Specialty Specific Factors.

14. The non-transitory computer-readable storage medium according to claim 11, wherein
performing the risk determination cycle occurs in response to an electronic request that is received at the interface from an apparatus, configured as a node on a remote network, that is operated by one or more of an insurer, a government entity, a prospective patient, a prospective employer, and a journalist, and
the method further comprises transmitting, by the processor, the strength of the likelihood of the possible adverse event or complication through the communication interface to the apparatus operated by the one or more of the insurer, the government entity, the prospective patient, the prospective employer, and the journalist.

15. The non-transitory computer-readable storage medium according to claim 14, wherein
the electronic request is received from the insurer, and
the method further comprises, after transmitting the strength of the likelihood of the possible adverse event or complication to the apparatus operated by the insurer:
calculating, by the processor, a malpractice premium for the particular physician or the particular medical institution based on the strength of the likelihood of the possible adverse event or complication, and
transmitting, by the processor through the communication interface, the malpractice premium to the apparatus operated by the insurer.
